(19) United States
(12) Patent Application Publication
(10) Pub. No.: US 2022/0089560 A1
(43) Pub. Date: Mar. 24, 2022

(54) Compositions of &#916;10-THC and &#916;6a10-THC

(71) Applicants: Alexander William Siegel, Albany, CA (US); Devin Jay Wall, Vallejo, CA (US); Alek Enrique Valle, Novato, CA (US)

(72) Inventors: Alexander William Siegel, Albany, CA (US); Devin Jay Wall, Vallejo, CA (US); Alek Enrique Valle, Novato, CA (US)

(21) Appl. No.: 17/028,824

(22) Filed: Sep. 22, 2020

(51) Int. Cl. C07D 311/80; C07C 39/21; C07C 39/19; C07C 37/84; C07C 37/06

(52) U.S. Cl. CPC C07D 311/80; C07C 39/21; C07C 37/06; C07C 37/84; C07C 39/19

(57) ABSTRACT

Compositions of Δ10-THC and Δ6a10a-THC are prepared and described. The conversions do not affect existing CBD or CBG in the extract. Various adjustments can be made to the reactions resulting in increased or decreased product and by-product. Various formulations with medical uses are disclosed.

### TECHNICAL FIELD

The present invention relates to the isolation and subsequent formulation of an extract of cannabis. More specifically, the present invention relates to the formulation of Δ10-THC and Δ6a10a-THC. Invention formulates the compounds into novel medicinal and recreational mixtures.

### BACKGROUND

Public interest in Cannabis as a medicine is well-established, based in no small part on the fact that Cannabis has long been considered to have medicinal properties, ranging from treatment of cramps, migraines, convulsions, appetite stimulation and attenuation of nausea and vomiting. In fact, a report issued by the National Academy of Sciences' Institute of Medicine indicated that the active components of Cannabis appear to be useful in treating pain, nausea, AIDS-related weight loss, muscle spasms in multiple sclerosis as well as other problems. Advocates of medical marijuana argue that it is also useful for glaucoma, Parkinson's disease, Huntington's disease, migraines, epilepsy and Alzheimer's disease.

There are many cannabinoids which do not have psychoactivity but have beneficial effects, including anti-anxiety, anti-inflammation, anti-fungal and more. Cannabinoids such as CBD, CBC, CBG, and CBN contain little or no psychoactivity and yet each have potential benefits not tied to the intoxicating effects of Δ9-THC

In 1988, Srebnick studied the psycho-activity of stereoisomers of Δ6a10a-THC and Δ10-THC in isolation in order to discriminate their effects. In the study, titled “Separation of the discriminative stimulus effects of stereoisomers of Δ2- and Δ3-tetrahydrocannabinols in pigeons” it was found that the R isomer of both Δ6a10a-THC and Δ10-THC are the least psychoactive, with almost no psycho-activity. Since the invention produces the same stereochemistry at that carbon, as shown by HNMR data included, it can be said with confidence that the Δ6a10a-THC and Δ10-THC produced is void of psycho-activity and can be formulated into non-psychoactive formulations.

Clearly, as the cannabinoids are of potential medicinal value, formulations containing these compounds are beneficial to study and manufacture.

### SUMMARY OF THE INVENTION

A method is disclosed for converting Δ9-THC to a non-psychoactive THC comprising:

1. Adding a catalyst to Δ9-THC.

2. Heating the reaction above 130° C.

3. Formulating purified non-psychoactive material into end products.

Varying the heat, catalyst type and amount, and the atmospheric conditions may result in the acceleration or deceleration of general and/or side reactions, which may create different amounts of non-psychoactive THC. The disclosed method generally may not alter other cannabinoids such as Δ8-THC, CBD, or CBG, which creates novel formulations of Δ6a10a-THC and Δ10-THC in combination with these compounds and others. Some of the most notable important formulations which can be manufactured currently are included. Additionally, some formulations may be made by intentionally combining and formulating isolated cannabinoids with Δ10-THC or Δ6a10a-THC. Potential medical uses are discussed.

### BRIEF DESCRIPTION OF DRAWINGS

FIG. 1 shows the chemical formula of the general reaction.

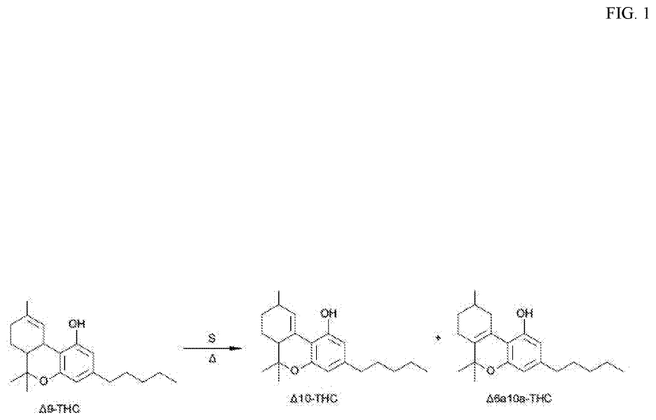

### DETAILED DESCRIPTION

In the Summary above and in this Detailed Description, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used—to the extent possible—in combination with and/or in the context of other particular aspects and embodiments of the invention, and in the invention generally

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range including that number (which may be a range having an upper limit or no upper limit, depending on the variable being defined). For example, “at least 1” means 1 or more than 1. The term “at most” followed by a number is used herein to denote the end of a range, including that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limits include both numbers. For example, “25 to 100” means a range whose lower limit is 25 and upper limit is 100, and includes both 25 and 100.

Described herein are methods and protocols for converting Δ9-THC to non-psychoactive cannabinoid, specifically 6aR,9R-Δ10-THC and 6aR-9R-Δ6a10a-tetrahydrocannabinol. When the spectra are compared to literature (Base-Catalyzed Double-Bond Isomerizations of Cannabinoids) we can confirm the presence of these reportedly non-psychoactive compounds. As will be appreciated by one knowledgeable in the art and as discussed below, the reaction times may be varied somewhat, producing product at different yields and purities. Furthermore, functional equivalents may be substituted where appropriate. Also described herein is the formulation of Δ10-tetrahydrocannabinol (Δ10-THC) and Δ6a10a-tetrahydrocannabinol (Δ6a10a-THC) with other relevant cannabinoids.

Yield may be determined by looking at the peak area for the isolated compound in the liquid chromatography—PDA analysis of the crude reaction product mixture.

Purity may also be determined by looking at the peak area for the isolated compound in the liquid chromatography—PDA analysis of the crude reaction product mixture or formulations.

One aspect of invention consists of adding A 9-THC in a reaction mixture; mixing the reaction mixture; heating the reaction mixture while mixing, adding catalyst, heating for a period of time; and allowing the reaction mixture to cool. Reactions that may be considered are listed below:

1. General Reaction 1: Δ9-THC+S (catalyst)+Δ→Δ10-THC
2. General Reaction 2: Δ10-THC+S (catalyst)+Δ→Δ6a10a-THC
3. Side Reaction 1: Δ9-THC+O₂+Δ→CBN+2 H₂O
4. Side Reaction 2: Δ10-THC+O₂+Δ→CBN+2 H₂O
5. Side Reaction 3: Δ6a10a-THC+O₂+Δ→CBN+2 H₂O

### 6. Side Reaction 4: Δ9-THC+2S+Δ→CBN+2 H₂S

### 7. Side Reaction 5: Δ10-THC+2S+Δ→CBN+2 H₂S

8. Side Reaction 6: Δ6a10a-THC+2S+Δ→CBN+2 H₂S

### 9. Side Reaction 7: Δ9-THC+Δ→CBN+2H₂

10. Side Reaction 8: Δ9-THC+Δ+H⁺→Δ8-THC+H⁺
Note that when concentration of Δ9-THC is decreased, all reaction rates decrease except for General Reaction 2, Side Reaction 2, Side Reaction 3, Side Reaction 5, and Side Reaction 6. When temperature is increased all reactions increase in rate. The reaction of Δ6a10a-THC converting into CBN is by far the fastest reaction when exposed to heat, oxygen, or catalyst, which minimizes its concentration in many samples. Conversion of Δ6a10a-THC may be a simple way to selectively decrease the Δ6a10a-THC in a mixture by converting it into CBN via oxidation. Some formulations which appear to have no Δ6a10a-THC may be produced, though they likely contain trace amounts.

### Example 1: Conversion of High Purity Δ9-THC to Δ10-THC, Δ6a10a-THC, and CBN Using Sulfur

The first example shows the General Reaction 1 and General Reaction 2 and uses nitrogen to minimize Side Reactions 1, 2, and 3 which generate excess CBN. Additionally, minimal sulfur was used in order to minimize Side Reactions 4, 5, and 6, which reduce concentration of Δ10-THC and Δ6a10a-THC. A higher temperature than Example 2 was used in order to increase the rate of General Reaction 1, generating a higher concentration of Δ10-THC, although Δ6a10a-THC may decrease due to degradation. A ratio of 22 grams of Δ9-THC (initially 25 gs of Δ9-THCa, 93.2% initial concentration) to 0.204 g elemental sulfur pellets was used although Δ9-THC concentration and catalyst concentration may vary. In this example, the mixture was heated to 200° C. under nitrogen and then catalyst was added. The mixture was heated under nitrogen for 1 hour. 5 minutes after the catalyst was introduced at 200° C., Δ9-THC concentration reduced to 41.3% with generation of 27.8% Δ10-THC, 20.7% Δ6a10a-THC, 4.20% CBN, and 0.7% Δ8-THC. 10 minutes after the catalyst was introduced at 200° C., Δ9-THC concentration reduced to 30.7% with generation of 34.8% Δ10-THC, 25.1% Δ6a10a-THC, 4.50% CBN, and 0.8% Δ8-THC. 20 minutes after the catalyst was introduced at 200° C., Δ9-THC concentration reduced to 21.3% with generation of 43.2% Δ10-THC, 27.2% Δ6a10a-THC, 5.20% CBN, and 1.0% Δ8-THC. 40 minutes after the catalyst was introduced at 200° C., Δ9-THC concentration reduced to 12.2% with generation of 51.1% Δ10-THC 23.4% Δ6a10a-THC, 6.2% CBN, and 1.1% Δ8-THC. 60 minutes after the catalyst was introduced at 200° C., Δ9-THC concentration reduced to 9.3% with generation of 55.5% Δ10-THC 20.3% Δ6a10a-THC, 6.90% CBN, and 1.2% Δ8-THC. 60 minutes after the catalyst was introduced at 200° C., the mixture was heated to 220° C. to investigate effect of higher heat. After 20 minutes ramping to 220° C., Δ9-THC concentration reduced to 8.1% with generation of 55.5% Δ10-THC, 17% Δ6a10a-THC, 7.0% CBN, and 1.2% Δ8-THC. After 20 minutes at 220° C., Δ9-THC concentration reduced to 6.1% with generation of 58.3% Δ10-THC, 14% Δ6a10a-THC, 8.1% CBN, and 1.2% Δ8-THC. The mixture was then cooled under nitrogen. Reversed-phase or normal-phase chromatography may be used to further purify Δ10-THC over 80% (Δ10-THC 85%, Δ6a10a-THC 14%). Another method of achieving high purity is through recrystallization of Δ10-THC in organic solvents, which may raise Δ10-THC purity above 90%. This is discussed in example 2.

### Example 2: Isolation of High Purity Δ10-THC and Δ6a10a-THC Through Recrystallization in a Solvent

A mixture containing Δ10-THC and Δ6a10a-THC was placed into solvent and recrystallized at room temperature, filtered to remove excess solvent, and dried. The solvent may be nearly any organic solvent where Δ10-THC is soluble, though non-polar solvents are most convenient. The purified mixture containing Δ10-THC and Δ6a10a-THC was 95% pure by HPLC PDA, with around 80% Δ10-THC and 15% Δ6a10a-THC. When the mixture contains Δ9-THC, CBN, or any other mixture of cannabinoids, there is usually some amount of cannabinoid contamination in the purified mixture, though generally minor. Recrystallization may be done under cold or warm conditions. The purified mixture containing Δ10-THC and Δ6a10a-THC yielded 50% with purity.

### Example 3: Conversion of Δ9-THC to Δ10-THC in Low Δ9-THC Material with CBD (Notebook B01-58.1)

The third example displays the General Reaction 1 and General Reaction 2 along with Side Reaction 4, 5, and 6 taking place, due to addition of excess catalyst. Additionally, higher temperatures than Example 3 was used in order to encourage General Reactions to complete, reducing Δ9-THC concentration below 0.30% although Δ9-THC concentration, catalyst concentration, and temperature may vary. Low vacuum (400,000 micron) was used in order to minimize Side Reactions 1, 2, and 3. A ratio of 30.4 grams of THC-sparse oil (4.1% Δ9-THC, 0.8% CBN, 56% CBD) to 0.2 grams (6.25 mmol) elemental sulfur pellets was used. In this example, the catalyst was added at room temperature and mixture was heated to 220° C. The mixture was heated under low vacuum for 1 hour. After 20 minutes under heat, Δ9-THC concentration reduced to non-detect with final concentrations of 1.4% Δ10-THC, 0.3% Δ6a10a-THC, and 3.0% CBN while maintaining original CBD content of 55.3% CBD. The increased temperature maintains the rate of the General Reaction until completion (Δ9-THC concentration is less than 30 mg/g) in less than 1 hour.

### Example 4: Conversion of Δ9-THC to Δ10-THC in Low Δ9-THC Material with Δ8-THC

The fourth example displays the General Reaction 1 and General Reaction 2 along with Side Reaction 4, 5, and 6 taking place, due to addition of excess catalyst. Additionally, higher temperatures than Example 3 was used in order to encourage General Reactions to complete, reducing Δ9-THC concentration below 0.30% although Δ9-THC concentration, catalyst concentration, and temperature may vary. Nitrogen was used in order to minimize Side Reactions 1, 2, and 3. A ratio of 50 grams of Δ9-THC-sparse oil (2.0% Δ9-THC, 0.8% CBN, 80% Δ8-THC) to 0.33 grams (10.3 mmol) elemental sulfur pellets was used. In this example, the catalyst was added at room temperature and mixture was heated to 200C. The mixture was heated under nitrogen for 1 hour. After 30 minutes under heat, Δ9-THC concentration reduced to non-detect with final concentrations of 1.1% Δ10-THC, 0.3% Δ6a10a-THC, and 1% CBN while maintaining original Δ8-THC content of 79.9% Δ8-THC. The increased temperature maintains the rate of the General Reaction until completion (Δ9-THC concentration is less than 30 mg/g) in less than 1 hour.

### Example 5: Conversion of Δ9-THC to Δ10-THC in Low Δ9-THC Material with CBG

The fifth example displays the General Reaction 1 and General Reaction 2 along with Side Reaction 4, 5, and 6 taking place, due to addition of excess catalyst. Additionally, higher temperatures than Example 3 was used in order to encourage General Reactions to complete, reducing Δ9-THC concentration below 0.30% although Δ9-THC concentration, catalyst concentration, and temperature may vary. Nitrogen was used in order to minimize Side Reactions 1, 2, and 3. A ratio of 51 grams of Δ9-THC-sparse oil (3.0% Δ9-THC, 0% CBN, 75% CBG) to 0.33 grams (10.3 mmol) elemental sulfur pellets was used. In this example, the catalyst was added at room temperature and mixture was heated to 185C. The mixture was heated under low vacuum for 1 hour. After 30 minutes under heat, Δ9-THC concentration reduced to non-detect with final concentrations of 1.3% Δ10-THC, 0.2% Δ6a10a-THC, and 2% CBN while maintaining most of the original CBG content of 49.2% CBG. The decreased temperature maintains the rate of the General Reaction until completion (9-THC concentration is less than 30 mg/g) without degrading CBG.

### Novel Formulations of Tetrahydrocannabinol

The effects of THC result from its partial agonist activity at the cannabinoid receptor CB1 located mainly in the central nervous system, and the CB2 receptor, mainly expressed in cells of the immune system. The psychoactive effects of THC are primarily mediated by the activation of CB1 receptor in the brain, which result in a decrease in the concentration of the second messenger molecule cAMP through inhibition of adenylate cyclase. Listed below are the THC isomers available to us in high concentration in order of decreasing activity at the CB1 receptor:

1. Δ9-Tetrahydrocannabinol

2. Δ8-Tetrahydrocannabinol

3. Δ10-Tetrahydrocannabinol (R-Isomer), Δ6a10a-Tetrahydrocannabinol (R-isomer) Although Δ9-THC can provide a large range of effects, including pain mediation and psychoactive effects, there are potential medical uses for the isomers which are less active at the CB1 receptor. For example, because of its lower activity at CB1, Δ8-THC can be consumed in higher quantities than Δ9-THC, which allow it to be used in high concentration formulations without causing as large of psychoactive effect. Δ10-Tetrahydrocannabinol and Δ6a10a-Tetrahydrocannabinol, notably the R isomers of each, are the least psychoactive isomers of THC available to us to be made into consumer products. Therefore, it is an area of interest to formulate Δ10-Tetrahydrocannabinol and Δ6a10a-Tetrahydrocannabinol into medical products wherein the consumer wants to avoid the psycho-activity associated with Δ9-Tetrahydrocannabinol (CB1) while potentially benefitting from the effects of THC attributed to activity at the CB2 receptors or other receptors. It has been found that activation of cannabinoid receptor 2 (CB2) prevents colitis-associated colon cancer through myeloid cell deactivation upstream of IL-22 production, creating an immediate potential use for such THC's which should be studied more deeply.

Additionally, many of the effects of THC may be altered by combining the THC with another cannabinoid such as CBD, CBG, or CBN. These cannabinoids have mild psychoactive activity or no psychoactive activity (CB1 activity) making them suitable cannabinoids for formulations wherein psychoactivity needs to be minimized. Each cannabinoid has specific effects and could be used to modulate pharmacokinetics of the cannabinoid receptors at the cellular level.

Some cannabinoids such as CBD and THC also interact because they are metabolized by the same enzyme in the liver (CYP450) and have been shown to affect the metabolism of each other without directly effecting the receptor activity at all. After metabolism by this enzyme Δ9-THC becomes more active and able to cross the blood-brain barrier, which alters the target effects of the drug. Cannabinoid interaction with this enzyme pathway is thought to be influenced by route of administration.

### Cannabigerol

Contrary to the major psychoactive cannabinoid THC, cannabigerol antagonizes CB1 receptors and is both an alpha2-adrenoceptor agonist and moderate 5HT1A receptor antagonist. Cannabigerol displays CB1 and CB2 binding affinity and has been evaluated in laboratory models of colitis. CBG may be combined with a THC in order to modulate the effects of THC on the cell. CBG is not known to be metabolized by the CYP450 enzyme, making it a suitable option when metabolism of some other cannabinoids such as THC shouldn't be inhibited.

### Cannabidiol

Cannabidiol has low affinity for the cannabinoid CB1 and CB2 receptors, although it can act as an antagonist of CB1/CB2 agonists despite this low affinity. Cannabidiol may be an antagonist of GPR55, a G protein-coupled receptor and putative cannabinoid receptor that is expressed in the caudate nucleusand putamen in the brain. It also may act as an inverse agonist of GPR3, GPR6, and GPR12. CBD has been shown to act as a serotonin 5-HT1A receptor partial agonist. It is an allosteric modulator of the μ- and δ-opioid receptors. The pharmacological effects of CBD may involve PPARγ agonism and intracellular calcium release. In addition, CBD is metabolized by the cytochrome p450 enzyme in the liver and competes with THC when present at the same time, modulating activity and potentially improving certain medical effects of the THC. In 2018, cannabidiol was FDA-approved (trade name Epidiolex) for the treatment of two forms of treatment-resistant epilepsy.

### Cannabinol

CBN acts as a partial agonist at the CB1 receptors but has a higher affinity to CB2 receptors; however, it has lower affinities relative to THC. Therefore, it may be useful to modulate CB2 receptors in conjunction with a THC. In addition, CBN is metabolized by the cytochrome p450 enzyme in the liver and competes with THC at the same time, modulating activity and potentially improving medical effects of the THC.

### Cannabichromene

In vitro, CBC is not active at CB1 or CB2 receptors, but is an agonist of TRPA1 and less potently, an agonist of TRPV3 and TRPV4. Although associated with pain regulation, activation of the TRP proteins discussed should not create any adverse interaction with THC, making CBC a suitable addition to a formula when the balance of CB1 and CB2 receptor activity need not be disturbed. CBC is antimicrobial, antifungal, and is a liquid at room temperature, making it a suitable liquifying agent as well.

### Cannabidiol and Δ9-THC

Nabiximols is a specific Cannabis extract that was approved in 2010 as a botanical drug in the United Kingdom. Though it did not pass phase 3 trials for pain relief, Nabiximols has been studied for a wide range of potential effects. In New Zealand it is approved for use as an add-on treatment for symptom improvement in people with moderate to severe spasticity due to multiple sclerosis who have not responded adequately to other anti-spasticity medications.

### Acidic Derivatives:

CBDa, CBGa, and THCa all have very little data on direct activity in humans, however we find that there is sufficient evidence to warrant their use in formulation and future study for medical purposes.

Novel Formulations and their Manufacturing

The following formulations, later claimed in this application, are derived from reactions explained in Examples 1-5 of this application. The following section explains how each formulation is made.

1. Δ10-THC, Δ6a10a-THC, Δ9-THC, CBN
This formulation is made using Example 1

2. Δ10-THC, Δ6a10a-THC, CBN
This formulation is made using Example 1, where Δ9-THC is fully converted through a similar reaction using more catalyst, heat, or time.

3. Δ10-THC and Δ6a10a-THC
This formulation is made using Example 1 followed by Example 2

4. Δ10-THC and CBN
This formulation is made using Example 1 followed by Example 2 and then followed by oxidation of Δ6a10a-THC into CBN using sulfur catalyst, though oxygen heat and time may effectively do the same.

5. Δ10-THC, Δ6a10a-THC, CBN, CBD
This formulation is made using Example 3 when Δ9-THC is fully reacted

6. Δ10-THC, Δ6a10a-THC, CBN, CBD, and Δ9-THC
This formulation is made using Example 3 with only partial reaction, resulting in Δ9-THC remaining in the mixture

7. Δ10-THC, CBN, CBD
This formulation is made using Example 3 when Δ9-THC is fully reacted and then followed by oxidation of Δ6a10a-THC into CBN using sulfur catalyst, though oxygen heat and time may effectively do the same.

8. Δ10-THC, Δ6a10a-THC, CBN, and Δ8-THC
This formulation is made using Example 4 when Δ9-THC is fully reacted

9. Δ10-THC, Δ6a10a-THC, CBN, Δ8-THC, and Δ9-THC
This formulation is made using Example 4 with only partial reaction, resulting in Δ9-THC remaining in the mixture

10. Δ10-THC, CBN, and Δ8-THC
This formulation is made using Example 4 when Δ9-THC is fully reacted and then followed by oxidation of Δ6a10a-THC into CBN using sulfur catalyst, though oxygen heat and time effectively do the same.

11. Δ10-THC and Δ6a10a-THC, CBN, and CBG
This formulation is made using Example 5 when Δ9-THC is fully reacted

12. Δ10-THC, Δ6a10a-THC, CBN, CBG, and Δ9-THC
This formulation is made using Example 5 with only partial reaction, resulting in Δ9-THC remaining in the mixture

13. Δ10-THC, CBN, and CBG
This formulation is made using Example 5 when Δ9-THC is fully reacted and then followed by oxidation of Δ6a10a-THC into CBN using sulfur catalyst, though oxygen heat and time effectively do the same.

### Recombined Formulations

Following the preparation of Formulations 1-13, these formulations with addition of CBC can be created. The acidic cannabinoids CBDa, THCa, CBGa can all be reintroduced in isolate form as well. The process described in Example 2 allows for the recombination of high purity Δ10-THC and Δ6a10a-THC with these cannabinoids, as well as CBD, CBN, CBG, and other THC's.

1. The conversion of psychoactive THC to one or more non-psychoactive cannabinoids.

2. The method of claim 1 performed in the presence of at least one other cannabinoid besides said THC, without degrading said other existing cannabinoid.

3. The method of claim 1 performed in the presence of CBD, cannabigerol, or Δ8-THC, without degrading more than 75% of said CBD, cannabigerol, or Δ8-THC.

4. A composition comprising Δ10-THC and at least one other cannabinoid.

5. The composition of claim 4; wherein said other cannabinoid is Δ6a10a-THC.

6. The composition of claim 4; wherein said other cannabinoid is Δ8-THC.

7. The composition of claim 4; wherein said other cannabinoid is Δ9-THC.

8. The composition of claim 4; wherein said other cannabinoid is cannabinol.

9. The composition of claim 4; wherein said other cannabinoid is cannabidiol.

10. The composition of claim 4; wherein said other cannabinoid is cannabigerol.

11. The composition of claim 4; wherein said other cannabinoid is cannabichromene.

12. The composition of claim 5; wherein said other cannabinoid is cannabinol.

13. The composition of claim 5; wherein said other cannabinoid is Δ8-THC.

14. The composition of claim 8; wherein said other cannabinoid is Δ8-THC.

15. The composition of claim 12; wherein said other cannabinoid is Δ8-THC.

16. A composition comprising Δ6a10a-THC and at least one other cannabinoid.

17. The composition of claim 16; wherein said other cannabinoid is Δ8-THC.

18. The composition of claim 16; wherein said other cannabinoid is Δ9-THC.

19. The composition of claim 16; wherein said other cannabinoid is cannabinol.

20. The composition of claim 16; wherein said other cannabinoid is cannabidiol.

21. The composition of claim 16; wherein said other cannabinoid is cannabigerol.

22. The composition of claim 16; wherein said other cannabinoid is cannabichromene.

23. The conversion of claim 2; wherein said cannabinoid is an acidic cannabinoid.

24. The composition of claim 4; wherein said cannabinoid is an acidic cannabinoid.

25. The composition of claim 16; wherein said cannabinoid is an acidic cannabinoid.

26. The composition of claim 9; further comprising CBN.
